(19) United States
(12) Patent Application Publication
(10) Pub. No.: US 2025/0255926 A1
(43) Pub. Date: Aug. 14, 2025

(54) GINGEROL COMPOSITIONS AND RELATED METHODS

(71) Applicant: ThermoLife International, LLC, Signal Hill, CA (US)

(72) Inventors: Ronald KRAMER, Signal Hill, CA (US); Alexandros NIKOLAIDIS, Nea Kallikratia (GR)

(21) Appl. No.: 19/048,816

(22) Filed: Feb. 7, 2025

Related U.S. Application Data
(60) Provisional application No. 63/551,486, filed on Feb. 8, 2024.
(60) Provisional application No. 63/679,559, filed on Aug. 5, 2024.

(51) Int. Cl. A61K 36/9068; A61K 9/08; A61K 9/48; A61K 31/12; A61K 33/00; A61K 47/10; A61P 29/00; A61P 31/22

(52) U.S. Cl. CPC A61K 36/9068; A61K 9/08; A61K 9/4858; A61K 31/12; A61K 33/00; A61K 47/10; A61P 29/00; A61P 31/22

(57) ABSTRACT

A composition for human ingestion that provides improved bioaccesibility, solubility, and/or bioavailability of the extract of ginger is disclosed. The composition comprises an extract of ginger and a source of lithium. A method of improving bioaccesibility, solubility, and/or bioavailability of an extract of ginger is further disclosed, and the method comprises: providing the extract of ginger; and mixing a source of lithium with the ginger extract, thereby producing a water-soluble form of ginger extract. Also disclosed is a method of treating a gingerol-improvable condition that comprises administering to a subject 0.01-30,000 ml of a composition comprising an extract of ginger and a source of lithium. Further disclosed is a method of improving athletic performance in a subject that comprises administering to the subject 0.01-30,000 ml of a composition comprising an extract of ginger and a source of lithium prior to the subject exercising.

### CROSS REFERENCE TO RELATED APPLICATIONS

The present application claims the benefit of and priority to U.S. provisional patent application 63/551,486, filed Feb. 8, 2024, and to U.S. provisional patent application 63/679,559, filed Aug. 5, 2024, the entirety of the disclosures of which are hereby incorporated by this reference.

### FIELD OF THE INVENTION

The disclosure relates to a process for preparing ginger extracts with improved bioavailability.

### BACKGROUND OF THE INVENTION

The term “ginger” refers a variety of plants. The most common plant referred to as “ginger” is Zingiber officinale, which is a flowering plant whose rhizome, which colloquially is called “ginger root” or simply “ginger”, is widely used as a spice and a folk medicine. It is an herbaceous perennial which grows annual pseudostems (false stems made of the rolled bases of leaves) about one meter tall, bearing narrow leaf blades. The inflorescences bear flowers having pale yellow petals with purple edges and arise directly from the rhizome on separate shoots.

Apart from the officinale variety in the family Zingiberaceae, other subspecies of ginger include Beehive Ginger (Zingiber spectabile), Bitter Ginger (Zingiber zerumbet), and Myoga ginger (Zingiber myoga). The term “ginger” is also used to describe other species of plants, which are crepe ginger (Cheilocostus speciosus), Hidden Ginger (Curcuma petiolate), Butterfly Lily Ginger (Hedichium coronarium), Shell Ginger (Alpinia zerumbet), Dancing ladies ginger (Globba winitiii), yellow ginger (Hedychium flavescens), Red ginger (Alpinia purpurata), torch ginger (Etlingera elatior), Mango ginger (Curcuma Amada), Kahili ginger (Hedychium garderianum), Thai ginger (Alpinia galanga), Pineapple ginger (Tapeinochilos ananassae), Resurrection Lily (Kaempferia rotunda), Siam Tulip (Curcuma alismatifolia), and snap ginger (Alpinia calcarate).

Though the potential medicinal uses of ginger root with minimal modification, as a powder, and its extracts have long been known, use of ginger or ginger extracts in nutraceuticals or enriched-food products is limited due to poor bioavailability of the ginger extract compounds. Thus, the bioaccesibility, solubility, and bioavailability of ginger actives needs to be improved to fully realize the health improvement potential of ginger extracts.

### SUMMARY OF THE INVENTION

A composition comprising an extract of ginger and a source of lithium, which is suitable for human ingestion is described. The composition improves the bioaccesibility, solubility, and/or bioavailability of the extract of ginger. In some embodiments, the weight ratio of the source of lithium and the extract of ginger is between 1:100 and 100:1. In some aspects, the source of lithium is metallic lithium. In other aspects, the source of lithium is a charged form of lithium such as lithium hydroxide or lithium hydride. The composition may further comprise a dietary ingredient, a food additive, or a pharmaceutically acceptable additive. In some aspects, the pharmaceutically acceptable additive in the composition is a polyethylene glycol (PEG), for example PEG 400.

The composition may be in a variety of dosage forms, for example, in the form of a tablet, capsule, a solution, an elixir, a syrup, a tincture, a suspension, an emulsion, a mouthwash, a spray, a drop, an ointment, a cream, a gel, a paste, a suppository, a pessary, a cream, a gel, or a foam. Where the composition is a solution, the pH of the solution is 8-14. In some aspects, the concentration of active compounds in the composition is at least 1000 micromole/L. In a preferred embodiment, the composition is in the form of a softgel.

Also described in a method of treating a ginger treatable condition in a subject. The method comprises administering to the subject 0.01-30,000 ml or 0.1-30,000 ml of the above-described composition per day. In some implementations, the subject is administered 0.01-100 ml or 0.1-100 ml of the composition per day. In some aspects, the subject is administered the composition twice a day. In a preferred embodiment, the subject is administered the composition in a softgel formulation.

A method of improving athletic performance in a subject is described. The method comprises administering to the subject 0.01-30,000 ml, 0.1-30,000 ml, 0.01-100 ml, or 0.1-100 ml of the above-described composition prior to the subject exercising. In some aspects, the improvement in athletic performance manifests as reduced muscle soreness during and/or after exercise.

Further described is a method of improving bioaccesibility, solubility, and/or bioavailability of an extract of ginger. In a preferred embodiment, the source of ginger is total CO₂ liquid ginger extract. It should also be noted that the methods described herein could be applied to produce water soluble formulations of isolated ginger compounds, such as gingerol or shogaol. For purposes of doctrine of equivalents, manufacturing or usage for the purposes described in the patent therein using a single water-insoluble ginger active compound or a mixture of water insoluble ginger active compounds shall be considered to be equivalent to manufacturing or using the water-soluble total CO₂ ginger extract. The method comprises providing the extract of ginger; and mixing a source of lithium with the extract of ginger, thereby producing a water-soluble form of ginger extract. The source of lithium may be selected from the group consisting of: metallic lithium, lithium hydroxide or lithium hydride. None of metallic lithium, lithium hydroxide, and lithium hydride exist in nature; they are man-made chemicals. The weight ratio of the source of lithium and the extract of ginger mixed together is between 1:100 and 100:1, between 1:50 to 50:1, between 1:20 to 20:1, or between 1:10 to 10:1.

In some implementations, the method further comprises dissolving the source of lithium in water to produce a lithium solution, wherein the lithium solution is mixed with the ginger extract. For such implementations, the method further comprises mixing at least one non-aqueous liquid with the water-soluble form of ginger extract. In some aspects, the at least one non-aqueous liquid is selected from the group consisting of: a polyol, an alcohol, or an oil. In certain implementations, the at least one non-aqueous liquid comprises PEG, for example PEG 400.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Implementations will hereinafter be described in conjunction with the appended and/or included DRAWINGS, where like designations denote like elements.

FIGS. 1A-E depicts the generalized structure of gingerols (FIG. 1A), shogoals (FIG. 1B), paradols (FIG. 1C), zingerone (FIG. 1D), and zingiberene (FIG. 1E).

FIGS. 2A and 2B respectively depict the FTIR of a ginger alcohol extract and the water-soluble ginger extract produced in Example 1. The FTIR of FIG. 2A is reproduced from Karthickeyan, “Effect of nature based antioxidant from Zingiber officinale Rosc. on the oxidation stability, engine performance and emission characteristics with neem oil methyl ester.” Heat Mass Transfer, 2018, 54:3409-3420.

FIGS. 3A-3B are photographs illustrating a liquid ginger extract produced according to the disclosed method (FIG. 3A) and the water-solubility of the liquid product (FIG. 3B).

FIGS. 4A and 4B compare the water solubility of a liquid ginger extract first combined with 1 g potassium hydroxide and 1 g sodium hydroxide (FIG. 4A) and then adding to the solution 1 g lithium hydroxide (FIG. 4B). The latter combination corresponds to the disclosed method of preparing a water-soluble ginger extract.

FIG. 5 is a graph of a subject's blood C-reactive protein level over three weeks of ingesting the preparation produced in Example 1.

FIG. 6 is a photograph of a liquid ginger extract in 100 ml lithium ascorbate solution.

FIG. 7 is a photograph of 2 ml the water-soluble ginger extract produced according to Example 1 in 100 ml of water after 5 gram of citric acid added.

FIG. 8 is a photograph of a sticky semisolid compound produced from vacuum drying the water-soluble ginger extract produced according to Example 1.

FIGS. 9A-9F depict exemplary structures of gingerol gallate, shogaol gallate, paradol gallate, shogaol vanillate, paradol vallinate, and gingerol vanillate.

### DETAILED DESCRIPTION OF THE INVENTION

Detailed aspects and applications of the disclosure are described below in the drawings and detailed description of the disclosure. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant arts, that the present disclosure may be practiced without these specific details. It should be noted that there are many different and alternative configurations, devices, and technologies to which the disclosed disclosures may be applied. The full scope of the disclosures is not limited to the examples that are described below.

The singular forms “a,” “an,” and “the” include plural referents unless the context clearly dictates otherwise. Thus, for example, reference to “a step” includes reference to one or more of such steps.

As used herein, the term “about” refers to a deviation no more than 10% of the given value, for example a deviation of 8%, 5%, 3%, 2%, 1%, 0.5%, or 0.1% of the given value.

It will be understood that the term “ginger” refers to Zingiber officinale, along with other species that are colloquially described as a variety of ginger, including but not limited to Zingiber spectabile, Zingiber zerumbet, Zingiber myoga, Cheilocostus speciosus, Curcuma petiolate, Hedichium coronarium, Alpinia zerumbet, Globba winitiii, Hedychium flavescens, Alpinia purpurata, Etlingera elatior, Curcuma Amada, Hedychium garderianum, Alpinia galanga, Tapeinochilos ananassae, Kaempferia rotunda, Curcuma alismatifolia, and Alpinia calcarate.

As used herein, “pharmaceutically acceptable additive” or “additive” are terms used in their broadest sense. Particular implementations of the compositions described in this document may also comprise an additive (e.g. one of a solubilizer, an enzyme inhibiting agent, an anticoagulant, an antifoaming agent, an antioxidant, a coloring agent, a coolant, a cryoprotectant, dilutant, a hydrogen bonding agent, a flavoring agent, a flow agent, a plasticizer, a preservative, a sweetener, a thickener, and combinations thereof) and/or a carrier (e.g. one of an excipient, a lubricant, a binder, a disintegrator, a diluent, an extender, a solvent, a suspending agent, a dissolution aid, an isotonization agent, a buffering agent, a soothing agent, an amphipathic lipid delivery system, and combinations thereof). These additives may be solids or liquids, and the type of additive may be generally chosen based on the type of administration being used. Those of ordinary skill in the art will be able to readily select suitable pharmaceutically effective additives from the disclosure in this document. In particular implementations, pharmaceutically acceptable additives may include, by non-limiting example, calcium phosphate, cellulose, stearic acid, crosscarmelose cellulose, magnesium stearate, polyethylene glycol (PEG), and silicon dioxide. In certain implementations, the pharmaceutically acceptable additive in the composition comprises PEG 400.

As used in this document, “metallic lithium” refers to the elemental metal form of lithium, which is lithium metal in its neutral, uncharged form.

As used in this document, “pharmaceutically effective” is a phrase used in its broadest sense, including, by non-limiting example, effective in a clinical trial or for a specific patient. When used in a method claim, pharmaceutically effective will mean in a dose enough to achieve the claim's preamble.

As used in this document, “pharmaceutically acceptable” is a phrase used in its broadest sense and may describe ingredients of a pharmaceutical composition that meet Food and Drug Administration (FDA) standards, United States Pharmacopeial Standards (USP), US Department of Agriculture (USDA) standards for food-grade materials, commonly accepted standards of the nutritional supplement industry, industry standards, botanical standards, or standards established by any individual. These standards may delineate acceptable ranges of aspects of ingredients of a pharmaceutical composition such as edibility, toxicity, pharmacological effect, or any other aspect of a chemical, composition, or preparation used in implementations of a pharmaceutical composition.

The present disclosure relates to the preparation of ginger extracts resulting in improved solubility and dissolution and ginger extract compositions with improved bioaccesibility, solubility, and/or bioavailability of active compounds in ginger extracts.

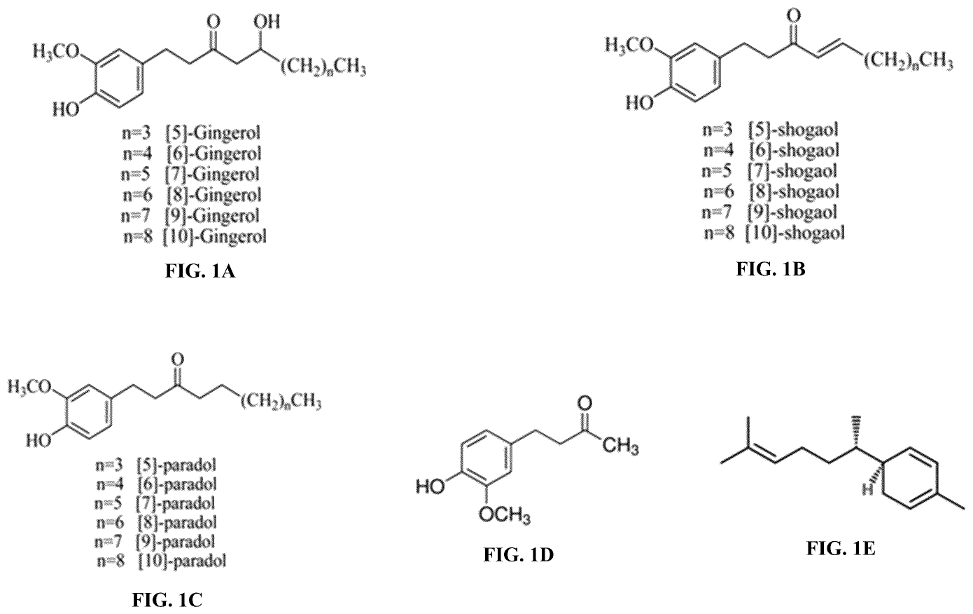

The scientific community has probed into the biological validation of ginger extracts through in vitro and in vivo studies and identified and isolated active compounds of ginger extracts. The isolated active compounds of ginger extracts include gingerols, shogaols, paradols, zingiberene, and zingerone. The structures of gingerols can be generalized in FIG. 1A. The structures of shogaols can be generalized in FIG. 1B. The structures of paradols can be generalized in FIG. 1C. The structure of zingerone is depicted in FIG. 1D. The structure of zingiberene is depicted in FIG. 1E.

Of known commercial preparations of ginger extracts, the total CO2 liquid ginger extract is the form of ginger extract with the highest percentage of active compounds and contains the most varieties of active compounds.

Table 1 depicts a typical analysis of a commercial total CO2 liquid ginger extract product.

TABLE 1

| Components | Range % | % |
|---|---|---|
| Content of essential oil | 30-50 | 35.1 |
| 6-Gingerol | 14-20 | 16.7 |
| Zingerone | <1 | 0.02 |
| 8-Gingerol | na | 4.1 |
| 6-Shogoal | na | 2.4 |
| 10-Gingerol | na | 6.1 |
| 8-Shogoal | na | 0.45 |
| 10-Shogoal | na | 0.20 |
| Sum of gingerols | na | 26.9 |
| Sum of shogoals | <5 | 3.1 |
| Sum of pungent compounds | 24-35 | 30 |
| Citral (neral/geranial)* | na | 4.6 |
| | | (1.7 + 2.9) |
| AR curcumene | na | 6.3 |
| α-Zingiberene | 20-45 | 34.5 |
| α-Farnesene | na | 8.5 |
| β-Bisabolene | na | 6.8 |
| Sesquiphellandrene | na | 13.3 |
| Methyl-eugenol | na | n.d. |
| | | (<0.01) |

*EU allergen
n.d. = not detected

Gingerols and derivatives are lipid soluble compounds and therefore it would be expected that they would have good absorption by passive diffusion across intestinal epithelium. However, prior to absorption, they must reach brush border cells, which implies that they are solubilized in an aqueous media. Due to their chemical structure, they present a very low solubility in water. Bioaccesibility is the first limiting step in whether or not a compound may exert an effect in an organism. This phenomenon is related to the concept of bioaccesibility, the amount of ingested nutrient available for absorption, which is different than the concept of bioavailability, which refers to the portion of the ingested dose of a compound that reaches the general circulation and specific sites where it can exert its action. Unfortunately, nutritional and clinical use of ginger in nutraceuticals or enriched-food products has been limited due to the poor bioavailability of the active compounds of ginger extracts. For example, the addition of the total CO2 liquid ginger extract to water results in virtually none of the extract being dissolved in water.

The solubility of various ginger phenolics has been described to be in the micromole (micromoles/L) range with pH conditions having minimal effect (Table 2).

TABLE 2

| GE | pH 2 - HCl | | pH 4 - citrate | | pH 7.4 - | |
|---|---|---|---|---|---|---|
| Phenolics | buffer | | buffer | | phosphate buffer | |
| 6-gingerol | >100 | μM | >100 | μM | >100 | μM |
| 8-gingerol | >100 | μM | >100 | μM | >100 | μM |
| 10-gingerol | <10 | μM | <10 | μM | <10 | μM |
| 6-shogoal | <10 | μM | <10 | μM | <10 | μM |

n = 2

Various methods have been employed to increase the bioaccesibility, solubility, and/or bioavailability of ginger actives. These technologies include nanoparticles, micelles, emulsions or solid dispersion liposomes or self-microemulsifying drug delivery systems. Studies performed in animal models on these forms of encapsulation of single and combined ginger compounds revealed somewhat improved pharmacokinetic profiles in most cases. However, there has been a lack of any remarkable commercial or scientific success of any of the above solutions.

Furthermore, there are foreseeable problems of these strategies for improving the bioaccesibility, solubility, and/or bioavailability of ginger extract. These problems include instability of such solutions, use of liquids such as water at amounts that prohibit inclusion in capsules, and the unpalatability of ginger for many people especially at higher concentrations of the pungent compounds, such as in the case of extracts. Highly concentrated ginger preparations have also been known to produce side effects, such as a burning feeling in mouth/throat, abdominal pain, diarrhea, or heartburn. The highly concentrated total CO2 liquid ginger extract is very irritating to the mouth and gastrointestinal tract when directly ingested. The doses of ginger provided in conventional supplement compositions are also typically very low in active compounds. As such, the amount of active compounds provided is insufficient to alter the body's natural processes/functions (i.e., suppress pain and inflammation, increase endurance, increase testosterone, increase nitric oxide, etc.). By comparison, 1000 mg of total CO₂ liquid ginger extract has the equivalent amount of active compounds contained in tens of kilograms of fresh ginger.

On the other hand, the methods, compositions, and formulations described in this disclosure are also surprisingly well tolerated (see the following Examples; no tester reported the onset of any uncomfortable side effects) and they provide high concentrations of the active ginger compounds. For example, 2 grams of a composition produced according to the methods described herein contain an amount of active ginger compounds equivalent to those found in about 3.6 kilograms of fresh ginger, which would be impossible for a human to tolerate and consume from a regular diet.

The method described in this disclosure for improving bioaccesibility, solubility, and/or bioavailability of ginger extracts comprises providing an extract of ginger and mixing a source of lithium with the ginger extract thereby producing a water-soluble form of ginger extract. For purposes of the doctrine of equivalents, manufacturing or usage for the purposes described herein using a single or a mixture of water insoluble ginger active compounds shall be considered as equivalent to manufacturing or using the water-soluble ginger extract.

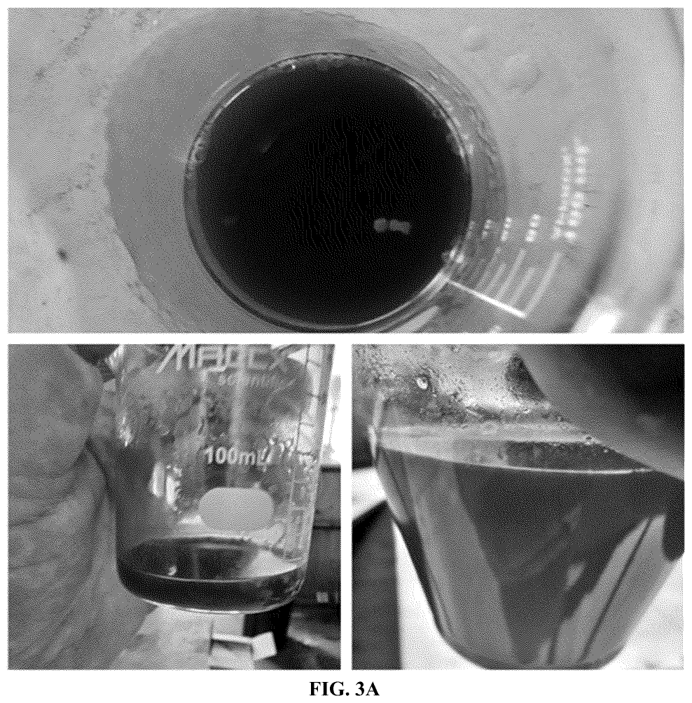

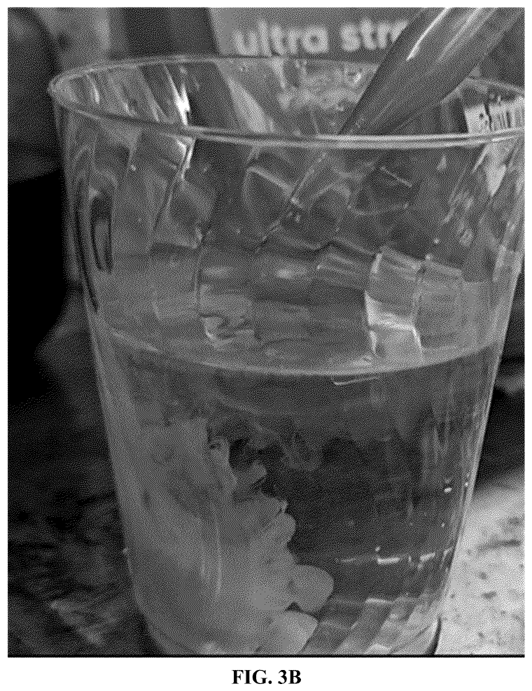

In a preferred embodiment, the source of ginger is total CO₂ liquid ginger extract. This form of ginger that has not been yet utilized in the supplement industry but contains a much more diverse number of actives at much higher concentrations than common extracts found is the market such as the ethanolic extract. As can be seen difference in the number of peaks between FIGS. 3A and 3B, the variety of active compounds in the ginger alcoholic extract (representative of common ginger extracts found in the market) is much lower compared to total CO₂ liquid ginger extract (albeit after reaction with a source of lithium).

It should also be noted that the methods described herein could be applied to produce water-soluble formulations of any isolated ginger compound, such as gingerol or shogaol.

The source of lithium may be selected from the group consisting of: metallic lithium, lithium hydroxide, and lithium hydride. None of metallic lithium, lithium hydroxide, and lithium hydride exist in nature; they are man-made chemicals.

One could theorize that metallic lithium, lithium hydroxide, and lithium hydride could be substituted for a lithium salt that is readily water soluble and result in an alkaline pH in solution (for example, salts of lithium with a weak acids like lithium citrate). Salts formed from a strong base and a weak acid are alkaline because when dissolved in water, the anion from the weak acid readily accepts a proton from water, generating hydroxide ions (OH⁻) and making the solution basic. Essentially, the weak acid part of the salt undergoes hydrolysis, producing more OH⁻ than H⁺, leading to an alkaline solution. In the exemplary case of lithium citrate, the citrate ion will readily react with water when in an aqueous solution to form unionized citric acid and hydroxide ions (OH⁻). For purposes of doctrine of equivalence, this would result in a similar alkaline solution comprising lithium and hydroxide ions. However as shown in Example 9, the use of lithium citrate, lithium lactate, or lithium ascorbate as the source of lithium did not result in any appreciable difference in solubility of the ginger extract. This may be to stereochemical blocking of the ginger compounds chemical moieties that interact with lithium. However, adding to the solution of lithium acid salt and ginger an excess of lithium (as Li, LiOH or LiH₂) results in the formation of a soluble complex. It is contemplated that removal of the anions from a lithium salt solution (such as with an ion exchange membrane or chemical conversion of the anion to a neutral or basic compound—for example nitrate to ammonia) will result in the formation of soluble complexes.

The weight ratio of the source of lithium and the extract of ginger mixed together is between 1:100 and 100:1, between 1:50 and 50:1, between 1:20 and 20 to 1, or between 1:10 and 10:1. In some implementations, the source of lithium and the extract of ginger is mixed with minimal amount of water—sufficient to achieve a saturated lithium hydroxide solution until a homogenous solution forms. The homogenous solution comprises the extract of ginger and the reacted form of the source of lithium and is usually colored orange to red.

For the preparation of softgels and capsules, the method further comprises diluting the homogenous solution with an additive to minimize the concentration of water so that the structure of gelatin needed for the softgel or capsule formulation is not disrupted. One method to reduce the water content would be to vacuum dry the water-soluble ginger extract until all water is removed. This results in the formation of a very sticky semisolid compound (FIG. 8). This compound is also water soluble albeit with a lower dissolution rate. In some aspects, the total concentration of water in the homogenous solution after dilution with the additive is less than 20%, less than 15%, less than 10%, or less than 5% by volume. The resulting solution is a water-soluble form of ginger extract. Components of the ginger extract and lithium ions can be detected and measured in the water-soluble form of ginger extract.

Preferably only a minimal amount of the source of lithium is mixed with the extract of ginger to provide water solubility to the ginger extract. Even more preferably, the amount of lithium is the minimal amount required to create a fully water-soluble ginger extract. In some aspects, for creating a fully dissolvable in water ginger extract, the weight ratio of the lithium element to the extract of ginger ranges from about 1:6000 to about 1:1, preferably about 1:600 to about 1:10, more preferably about 1:300 to about 1:20, even more preferably about 1:200 to about 1:40, still more preferably about 1:100 to about 1:50.

The resulting water-soluble form of ginger extract is at least partially soluble in water. For example, the solubility of the water-soluble form of ginger extract in water is at least about 1 mg/L or at least about 10 mg/L.

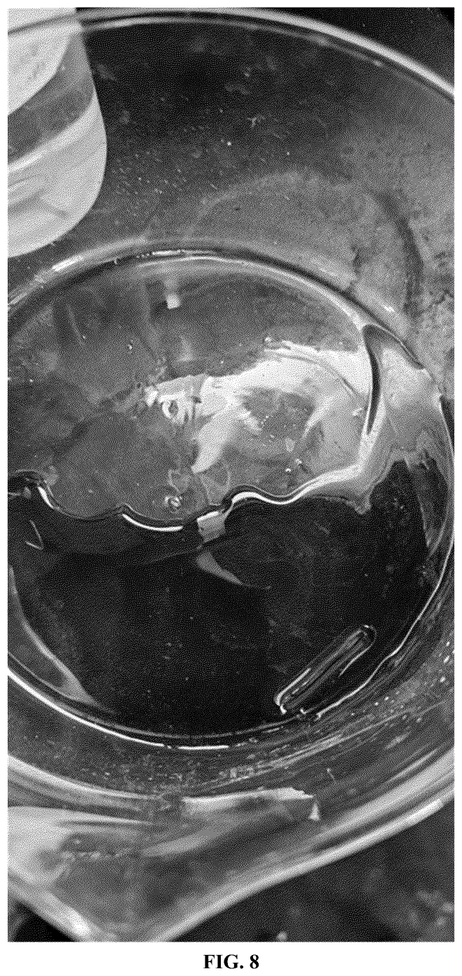

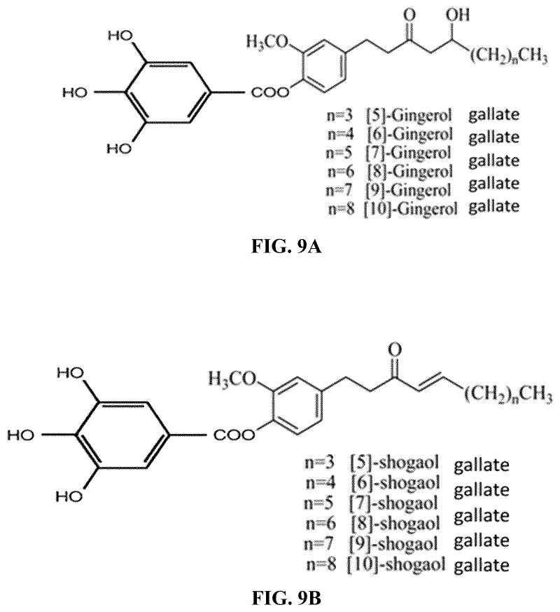

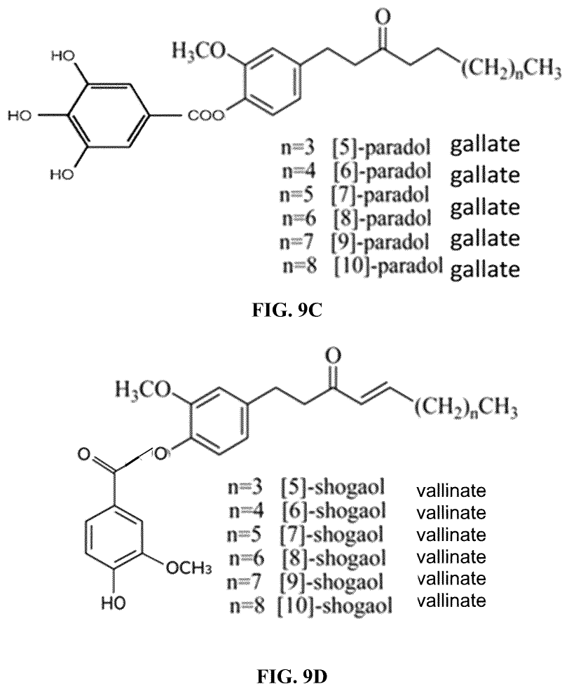

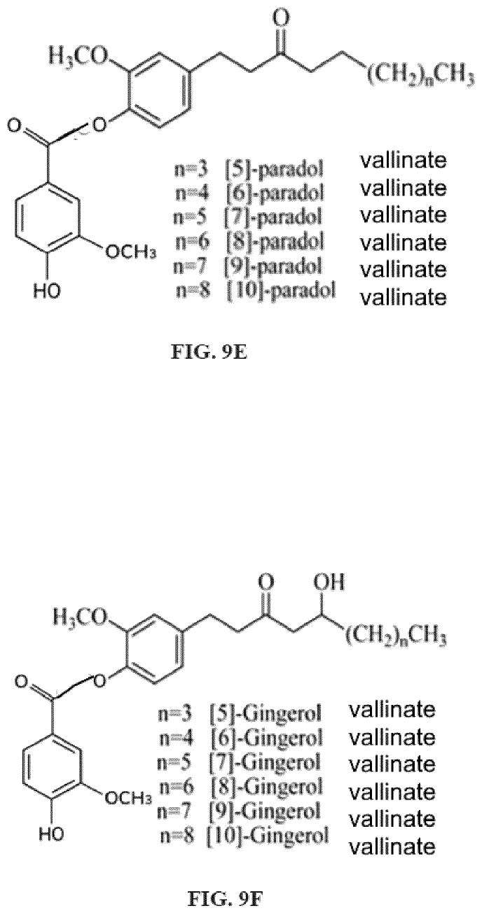

The disclosure also relates to compositions comprising an extract of ginger and a source of lithium, wherein the composition is a water-soluble form of ginger extract. It is of particular interest that the peak observed at 1255 nm in FIG. 3B, which denotes the presence of a phenolic ester, is not present in the FTIR of total CO₂ liquid ginger extract before reaction with the source of lithium. This is surprising, as alkaline compounds such as lithium hydroxide are known to break down ester bonds instead of facilitating the formation. Thus, the reaction of total CO₂ liquid ginger extract with the source of lithium yields new compounds. It is contemplated that these new compounds are esters of the phenolic compounds with gallic and cinnamic acid, which are naturally present in ginger. Thus, in some aspects, the composition comprises an ester of the phenolic compounds in ginger extract with gallic, vanillic, and cinnamic acid. For example, the composition comprises at least one of gingerol cinammate, gingerol gallate, gingerol vanillate, shogaol cinnamate, shogaol gallate, shogaol vanillate, paradol cinammate, paradol gallate, and paradol vanillate. FIGS. 9A-9F depict exemplary structures of some of the new compounds.

In some implementations, the composition further comprises one or more food or pharmaceutical additives. The additive is preferably a non-aqueous liquid, for example, a polyol, an alcohol, or an oil. In certain embodiments, the additive is propylene glycol, polyethylene glycol (for example PEG 400), glycerol, or ethanol.

The invention also contemplates formulations of said ginger solution with other ingredients that may synergistically work to treat any of the ginger treatable conditions.

Ingredients that may be included into ginger formulations to enhance its effects include but are not limited to1,3-DMAA; 1-androsterone; 1-epiandrosterone; 1,3-DMBA; 1,4-butanediol; 1,4-DMAA; 4-androsterone; 5-deca zol; 5-HTP; 5aOHP; 6-bromo-androstenedione; 7-methoxyflavone; 7,8-benzoflavone; abscess root; abuta; Acacia rigidula; acai; acerola; acetyl-L-carnitine or a salt, ester, or amide suitable for human ingestion thereof; ackee; aconite; activated charcoal; active hexose correlated compound (AHCC); adenosine; adrafinil; adrenal extract; adrue; aegeline; African wild potato; agar; agaricus mushroom; agave; agmatine or a salt, ester, or amide suitable for human ingestion thereof; agrimony; Ajuga nipponensis; alanine (including beta-alanine) or a salt, ester, or amide suitable for human ingestion thereof; Albizia julibrissin; Alchemilla; alder buckthorn; Aletris; alfalfa; algal oil; algin; alkanna; allspice; aloe; alpha lipoic acid; alpha-GPC; alpha-ketoglutarate (AKG); alpha-linolenic Acid (ALA); alpha-lipoic acid; alpine ragwort; Alpinia, amaranth; ambrette; American adder's tongue; American chestnut; American dogwood; American elder; American ginseng; American hellebore; American ivy; American mistletoe; American pawpaw; American spikenard; American white water lily; andarine; andiroba; Andrographis; androstenediol; androstenedione; androstenetrione; androsterone; Angel's Trumpet; Angelica archangelica; angostura; anhydrous crystalline maltose; aniracetam; anise; annatto; antineoplastons; antioxidants; apoaequorin; apple; apple cider vinegar; apple polyphenols; apricot; apricot kernel; arabinoxylan; arecoline; arenaria rubra; arimistane; Aristolochia; arnica; arrach; arrowroot; arsenic; Artemisia herba-alba, artichoke; arum; asafoetida; asarabacca; Ascophyllum nodosum; ascorbigen; ash; ashitaba; ashwagandha; Asian water plantain; asparagus (for example, Asparagus racemosus); aspartic acid or a salt, ester, or amide suitable for human ingestion thereof; aspen; astaxanthin; astragalus; Atlantic cedar; atractylodes; autumn crocus; avens; avocado; avocado soy unsaponifiables (ASU); avocado sugar extract; ayahuasca; ba ji tian; babassu; bach flower remedies; Bacillus coagulans; Bacopa monnieri; bael; Baikal skullcap; bamboo; banaba; banana; baobab; barley; basil; bay leaf; bayberry; bear's garlic; bee pollen; bee venom; beer; beeswax; beet; belladonna; benfotiamine; benzoin; berberine; bergamot; beta-carotene; beta-cryptoxanthin; beta-glucans; beta-hydroxybutyrate (BHB); beta-methylphenethylamine (BMPEA); beta-sitosterol; betaine anhydrous; betaine hydrochloride; betel nut; beth root; betony; Bifidobacterium animalis subsp. Lactis; Bifidobacterium bifidum; Bifidobacterium breve; Bifidobacterium longum; bilberry; biotin; birch; bishop's weed; bismuth; bismuth nitrate; bistort; bitter almond; bitter melon; bitter milkwort; bitter orange; bitter yam; bittersweet nightshade; black alder; black bryony; black cohosh; black currant; black haw; black hellebore; black hoof mushroom; black horehound; black mulberry; black mustard; black nightshade; black pepper; black psyllium; black raspberry; black rice; black root; black seed; black tea; black walnut; blackberry; blackthorn; blessed thistle; blond psyllium; bloodroot; blue cohosh; blue flag; blue-green algae; blueberry; bog bilberry; bog labrador tea; bogbean; bois de rose oil; boldo; boneset; borage; boron; Boswellia serrata; bovine cartilage; bovine colostrum; boxwood; branched-chain amino acids (BCAA); breadfruit; brewer's yeast; Brickellia, Bridelia, broccoli; broccoli sprout; bromelain; brown rice; brussels sprout; bryonia; buchu; buck's-horn plantain; buckhorn plantain; buckwheat; bugle; bugleweed; Bulbine natalensis; bulbous buttercup; Bupleurum; burdock; burning bush; burr marigold; butcher's broom; Butea superba; butterbur; buttercup; butternut; butylated hydroxytoluene (BHT); cabbage; cade; caffeic acid; caffeine; cajeput oil; calabar bean; calabash chalk; calamint; calamus; calanus oil; calcium; calcium d-glucarate; Calea zacatechichi; calendula; California poppy; Calotropis; calumba; camphor; camu camu; Canada balsam; Canadian fleabane; Canadian hemp; canaigre; cananga oil; cannabichromene (CBC); cannabidiol (CBD); cannabidivarin (CBDV); cannabigerol (CBG); cannabinol (CBN); cannabis; canola oil; canthaxanthin; capers; caprylic acid; capsicum; Caralluma; caraway; Carbon 60 (C60); cardamom; cardarine; carlina; carnitine or a salt, ester, or amide suitable for human ingestion thereof; carnosine or a salt, ester, or amide suitable for human ingestion thereof; carob; carqueja; carrageenan; carrot; cascara sagrada; cascarilla; casein peptide or protein; cashew; cassava; Cassia auriculata; Cassia cinnamon; Cassia nomame; cassie absolute; castor bean; castoreum; cat's claw; cat's foot; catechu; catnip; catuaba; cauliflower; Celastris paniculatus; celery; centaury; centrophenoxine; Cereus; cesium; cetylated fatty acids (CFAs); Ceylon cinnamon; Ceylon leadwort; cha de bugre; chaga; chanca Piedra; chaparral; chaulmoogra; cheken; chelation therapy products; chenopodium oil; chervil; chia; chickweed; chicory; Chinese cucumber; Chinese mallow; Chinese prickly ash; chirata; chitosan; chive; Chlorella; chlorine dioxide; chlorophyll; chlorophyllin; chokeberry; choline or a salt, ester, or amide suitable for human ingestion thereof, chondroitin sulfate; chromium; chrysanthemum; chrysin; chuchuhuasi; chymotrypsin; cilantro; cinchona; Cissus quadrangularis; Cistanche deserticola, citicoline; citric acid; citronella oil; clary sage; clay; Clematis recta; Clitoria ternatea; clivers; clove; clown's mustard plant; clubmoss; Cnidium; cobalt; coca; cocillana; cocoa; coconut; coconut oil; coconut water; cod liver oil; Codonopsis; coenzyme Q10; coffee; coffee charcoal; cola nut; Coleus; collagen type I (native) or type II (native) or peptides thereof; collard; colloidal minerals (for example, colloidal silver); colocynth; coltsfoot; columbine; coluracetam; Combretum micranthum; comfrey; common stonecrop; condurango; conjugated linoleic acid (CLA); Convolvulus pluricaulis; copaiba balsam; copper; coral; cordyceps; coriander; corkwood tree; corn poppy; corn silk; cornflower; Corydalis yanhusuo; costus; cotton; couch grass; cowhage; cowslip; cramp bark; cranberry; creatine or a salt, ester, or amide suitable for human ingestion thereof; croton seeds; cubebs; cucumber; cudweed; cumin; cursed buttercup; cyanostane; cyclamen; cypress; D-mannose; daffodil; damiana; dandelion; danshen; date palm; Datura wrightii; deanol; deer velvet; delta-8-tetrahydrocannabinol (Delta-8-THC); delta-9-tetrahydrocannabinol (THC); Dendrobium; desert parsley; devil's claw; devil's club; DHEA (for example, 19-nor-DHEA, 7-alpha-hydroxy-DHEA, 7-beta-hydroxy-DHEA, and 7-keto-DHEA); diacylglycerol; diatomaceous earth; diindolylmethane; diiodothyronine; dill; dimethylethanolamine (DMAE); dimethylglycine (DMG); dimethylhexylamine (DMHA); dimethylsulfoxide (DMSO); diosmin; divi-divi; docosahexaenoic acid (DHA); dodder; dolomite; dong quai; douglas fir; dragon fruit; dragon's blood; duckweed; dulse; durabolin; durian; dusty miller; dwarf elder; dwarf pine needle; dyer's broom; dymethazine; eastern hemlock; eastern red cedar; ecdysteroids; Echinacea; ecklonia cava; eicosapentaenoic acid (EPA); elderberry; elderflower; elecampane; elemi; eleuthero; ellagic acid; elm bark; emu oil; English adder's tongue; English horsemint; English ivy; English walnut; ephedra; epiandrosterone; epistane; equol; ergot; ergothioneine; eryngo; eucalyptus; Euphorbia cyparissias; Euphorbia hirta; European barberry; European buckthorn; European chestnut; European five-finger Grass; European mandrake; European mistletoe; Eurycoma longifolia; evening primrose; Evodia; eyebright; Fadogia agrestis; false unicorn; fennel; fenugreek; fermented milk; fermented wheat germ extract; fever bark; feverfew; ficin; field scabious; fig; figwort; fir; fireweed; fish oil; flaxseed; flaxseed oil; fluoride; fly agaric mushroom; fo-ti; folic acid; fool's parsley; forget-me-not; forskolin; forsythia; foxglove; frankincense; fringetree; frostwort; fructo-oligosaccharides (FOS); Fucus vesiculosus; fulvic acid; fumitory; galacto-oligosaccharides (GOS); galbanum; Galphimia glauca, gamboge; gamma butyrolactone (GBL); gamma-aminobutyric acid (GABA); gamma-hydroxybutyrate (GHB); gamma-linolenic acid (GLA); gamma-oryzanol; Garcinia, garden cress; gardenia; garlic; gelatin; Gelsemium; genistein combined polysaccharide; gentian; German chamomile; German ipecac; German sarsaparilla; germander; germanium; ginger; Ginkgo biloba, ginseng; globe flower; globemallow; glossy privet; glucomannan; glucosamine; glucuronolactone; glutathione; glutaurine; glycerol; glycine or a salt, ester, or amide suitable for human ingestion thereof; glycolic acid; glycomacropeptide; glyconutrients; goa powder; goat's rue; goji; golden ragwort; goldenrod; goldenseal; goldthread; gossypol; gotu kola; goutweed; grains of paradise; grape; grapefruit; gravel root; graviola; great plantain; greater burnet; greater celandine; Greek sage; green coffee; green tea; Griffonia simplicifolia; ground ivy; ground pine; groundsel; guaiac wood; guar gum; guarana; guarumo; guava; guggul; gum Arabic; gumweed; gymnema; halodrol-50; haronga; hartstongue; Hawaiian baby woodrose; hawthorn; hazelnut; heart's ease; heather; hedge mustard; hedge-hyssop; hemlock; hemlock water dropwort; hemp; hemp agrimony; hemp nettle; henbane; henna; herb Paris; herb Robert; Hercules club; hesperidin; hexadrone; hexylamine; Hibiscus sabdariffa; higenamine; histidine; holly; hollyhock; holy basil; homotaurine; honey; honeysuckle; hoodia; hops; hordenine; horny goat weed; horse chestnut; horsemint; horseradish; horsetail; hound's tongue; houseleek; hu zhang; humic acid; huperzine A; hyacinth bean; hyaluronic acid; hydrangea; hydrazine sulfate; hydroxymethylbutyrate (HMB); hyperimmune egg; hyssop; iboga; Iceland moss; idebenone; Ignatius bean; immortelle; Indian cassia; Indian gooseberry; Indian long pepper; Indian snakeroot; indigo pulchra; indium; indole-3-carbinol; inosine; inositol; inositol nicotinate; inulin; iodine; ip-6; ipecac; iporuru; ipriflavone; iron; Irvingia gabonensis; isatis; isopropylnorsynephrine; ivy gourd; jaborandi; jackfruit; jalap; Jamaican dogwood; jambolan; Japanese apricot; Japanese mint; Japanese persimmon; jasmine; java tea; Javanese turmeric; jequirity; jewelweed; jiaogulan; jimson weed; job's tears; jojoba; juniper; Justicia pectoralis; synthetic marijuana (for example, K2 or Spice); kale; kamala; kanna; kaolin; karaya gum; kava kava; kefir; ketogenic diet; khat; khella; kinetin; kiwi; knotweed; kohlrabi; kombucha; Korean pine; kousso; kratom; krill oil; kudzu; L-arginine or a salt, ester, or amide suitable for human ingestion thereof; L-carnitine or a salt, ester, or amide suitable for human ingestion thereof; L-citrulline or a salt, ester, or amide suitable for human ingestion thereof; L-cysteine or a salt, ester, or amide suitable for human ingestion thereof; L-glutamine or a salt, ester, or amide suitable for human ingestion thereof; L-ornithine or a salt, ester, or amide suitable for human ingestion thereof; L-aspartate or a salt, ester, or amide suitable for human ingestion thereof; L-phenylalanine or a salt, ester, or amide suitable for human ingestion thereof; L-theanine or a salt, ester, or amide suitable for human ingestion thereof; L-tryptophan or a salt, ester, or amide suitable for human ingestion thereof; L-tyrosine or a salt, ester, or amide suitable for human ingestion thereof; labdanum; laburnum; lactase; lactic acid; Lacticaseibacillus casei; Lacticaseibacillus paracasei; Lacticaseibacillus rhamnosus; Lactiplantibacillus pentosus; Lactiplantibacillus plantarum; Lactobacillus acidophilus; Lactobacillus crispatus; Lactobacillus delbrueckii; Lactobacillus gasseri; Lactobacillus helveticus; Lactobacillus jensenii; Lactobacillus johnsonii; lactoferrin; lady's bedstraw; laminaria; larch arabinogalactan; larch turpentine; lathyrus; Latilactobacillus sakei; laurelwood; lauric acid; lavender; lavender cotton; laxogenin; lecithin or a salt, ester, or amide suitable for human ingestion thereof; lemon; lemon balm; lemon eucalyptus; lemon verbena; lemongrass; lentinan; lesser celandine; levant berry; Levilactobacillus brevis; licorice; ligandrol; Ligilactobacillus salivarius; lily-of-the-valley; lime; limonene; Limosilactobacillus fermentum; Limosilactobacillus reuteri; linden; lingonberry; lion's mane mushroom; lipase; lithium; liver extract; liverwort; lobelia; logwood; Lorenzo's oil; lotus; lousewort; lovage; luffa; lunasin; lungmoss; lungwort; lupin; lutein; lychee; lycopene; lysine; m1-4add; maca; macadamia nut; mace; Madagascar periwinkle; madder; magnesium; magnolia; maidenhair fern; maitake mushroom; Malabar nut; male fern; malic acid; mallow; manaca; Manchurian thorn; manganese; mangosteen; manna; maqui; maral root; maritime pine; marjoram; marsh blazing star; marsh labrador tea; marsh marigold; marshmallow; masterwort; mastic; meadowsweet; medium chain triglycerides (MCTs); melanotan; melatonin; mentabolan; mercury herb; mesoglycan; methasterone; methionine; methoxydienone; methoxylated flavones; methyldiazinol; methylstenbolone; methylsulfonylmethane (MSM); methylsynephrine; mezereon; milk thistle; miracle fruit; molybdenum; moneywort; monolaurin; monterey pine; moringa; mormon tea; motherwort; mountain ash; mountain flax; mountain laurel; mouse-ear hawkweed; mugwort; muira puama; mullein; musk; myrcia; myrrh; myrtle; n-acetyl cysteine (NAC) or a salt, ester, or amide suitable for human ingestion thereof; N-acetyl-L-tyrosine or a salt, ester, or amide suitable for human ingestion thereof; N-methyltyramine; N,N-DMPEA; NADH; nasturtium; nattokinase; neem; nefiracetam; nerve root; New Jersey tea; New Zealand green-lipped mussel; niacin or a salt, ester, or amide suitable for human ingestion thereof; niacinamide; niauli oil; nickel; nicotinamide riboside; nicotine; nikko maple; noni; noopept; northern prickly ash; Norway spruce; nutmeg; nux vomica; oak moss; oats; oatstraw; octacosanol; octopamine; oleander; oleic acid; olive; olive oil; omega-6 fatty acids; onion; oolong tea; orchic extract; oregano; Oregon grape; oriental arborvitae; ornamental marigold; ornithine; ornithine ketoglutarate (OKG); orris; Oscillococcinum®; osha; ostarine; ostrich fern; oswego tea; ox-eye daisy; oxiracetam; padang cassia; pagoda tree; palm oil; palmitoylethanolamide (PEA); Panax ginseng; Panax notoginseng; pancreatic enzyme products; pangamic acid; pantethine; pantothenic acid or a salt, ester, or amide suitable for human ingestion thereof; pao pereira; papain; papaya; para-aminobenzoic acid (PABA); pareira; parsley; parsley piert; parsnip; partridgeberry; passion flower; pata de vaca; patchouli oil; pau d′arco; pea protein; peanut oil; pear; pectin; pedunculate oak; pellitory; pellitory-of-the-wall; pennyroyal; peony; peppermint; perilla; perillyl alcohol; periwinkle; Peru balsam; peyote; Phaseolus vulgaris; pheasant's eye; Phellodendron; phenethylamine (PEA); phenibut; phenpromethamine; phenylalanine or a salt, ester, or amide a salt, ester, or amide suitable for human ingestion thereof; phenylpiracetam; phlorizin; phosphate salts; phosphatidylcholine; phosphatidylserine; phytase; picamilon; Picrorhiza; Pimpinella; pine bark extract; Pinellia ternata; pink root; pipsissewa; piracetam; pitcher plant; plant sterols; pleurisy root; plum; Podophyllum; Poinsettia; poison ivy; pokeweed; polarity therapy; policosanol; polydextrose; Polypodium leucotomos; pomegranate; poplar; poppy seed; poria mushroom; potassium; potato; Potentilla, pramiracetam; pregnenolone; premorse; prickly pear cactus; procaine; progesterone; proline or a salt, ester, or amide a salt, ester, or amide suitable for human ingestion thereof; propionyl-l-carnitine or a salt, ester, or amide suitable for human ingestion thereof; propolis; proteolytic enzymes (proteases); psilocybin; pterostilbene; pu-erh tea; Pulsatilla; pumpkin; purple loosestrife; purple nut sedge; pygeum; Pyrethrum; pyrroloquinoline quinone (PQQ); pyruvate; quassia; quebracho blanco; queen's delight; quercetin; quillaia; quince; quinoa; Rabdosia rubescens; radish; raspberry ketone; Rauvolfia vomitoria, rauwolscine; red clover; red maple; red raspberry; red sandalwood; red soapwort; red yeast rice; red-spur valerian; reduced nicotinamide adenine dinucleotide (NADH); reed herb; Rehmannia; reishi mushroom; resveratrol; rhatany; Rhodiola; Rhodiola rosea; rhubarb; riboflavin; ribose; rice bran; rice bran arabinoxylan compound; rice protein, RNA, and DNA; rock rose; Roman chamomile; rooibos; rose geranium oil; rose hip; rosemary; royal jelly; rue; rupturewort; rusty-leaved rhododendron; rutin; rye grass; s-23; s-adenosyl methionine (SAMe); Saccharomyces boulardii; safed musli; safflower; saffron; sage; saigon cinnamon; salacia; Salatrim (short- and long-chain acyl triglyceride molecule); salep; Salvia divinorum; samphire; sandy everlasting; sangre de grado; sanicle; sarsaparilla; sassafras; savin tops; saw palmetto; scarlet pimpernel; Sceletium; Schisandra, schizandrol-A; Schizonepeta; Scopolia; Scotch broom; Scotch thistle; scurvy grass; sea buckthorn; sea moss; secretin; Securinega suffruticosa; selenium; self-heal; senega; senna; serine; serrapeptase; sesame; sessile oak; shark cartilage; shark liver oil; shea butter; shellac; shepherd's purse; shiitake mushroom; Siberian cocklebur; Sida cordifolia; silicon; Simaruba; sitostanol; skullcap; skunk cabbage; slippery elm; smartweed; smooth alder; snake skin; sodium; sodium bicarbonate; sodium tetrachloroaurate; solomon's seal; sorghum; sorrel; soy; soybean oil; Spanish broom; Spanish origanum oil; spearmint; spinach; spiny restharrow; spleen extract; spotted geranium; squalamine; squill; St. John's wort; star anise; star of Bethlehem; stavesacre; stenabolic; Stereospermum; stevia; stinging nettle; stone root; storax; strawberry; Streptococcus thermophilus; strontium; Strophanthus; succinate; sulbutiamine; sulforaphane; sulfur; suma; sumbul; summer savory; sundew; sunflower oil; superoxide dismutase (SOD); swallowroot; swamp milkweed; sweet almond; sweet Annie; sweet cherry; sweet cicely; sweet clover; sweet gale; sweet orange; sweet sumac; sweet vernal grass; sweet violet; sweet woodruff; syrian rue; tamarind; Tamarix dioica; tangerine; tannic acid; tansy; tansy ragwort; tapioca; tarragon; tart cherry; tartaric acid; taurine or a salt, ester, or amide suitable for human ingestion thereof; tea tree oil; teazle; Terminalia; testolone; tetrahydrocannabivarin (THCV); theacrine or a salt, ester, or amide suitable for human ingestion thereof; theaflavin; theanine; thiamine or a salt, ester, or amide a salt, ester, or amide suitable for human ingestion thereof; threonine or a salt, ester, or amide suitable for human ingestion thereof; thuja; thunder god vine; thyme; thymus extract; thyroid extract; tianeptine; timothy grass; tin; Tinospora cordifolia; tiratricol; tocotrienols; tolu balsam; tomato; tonka bean; toothed clubmoss; tormentil; tragacanth; trailing arbutus; transfer factor; traveler's joy; tree of heaven; tree tobacco; tree turmeric; trendione; tribulus; Trichopus zeylanicus; Tronadora; trypsin; tung seed; turkey corn; turkey tail mushroom; turmeric; turpentine oil; turtlehead; Tylophora; tyramine; tyrosine; ubiquinone (for example, coenzyme q10); umckaloabo; uridine; Usnea; uva ursi; uzara; valerian; vanadium; vanilla; verbena; veronica; vetiver; vietnamese coriander; vinpocetine; vitamin A or a salt, ester, or amide suitable for human ingestion thereof; vitamin B1 or a salt, ester, or amide suitable for human ingestion thereof; vitamin B12 or a salt, ester, or amide suitable for human ingestion thereof; vitamin B2 or a salt, ester, or amide suitable for human ingestion thereof; vitamin B6 or a salt, ester, or amide suitable for human ingestion thereof; vitamin B8 or a salt, ester, or amide suitable for human ingestion thereof, vitamin B9 or a salt, ester, or amide suitable for human ingestion thereof; vitamin C or a salt, ester, or amide suitable for human ingestion thereof; vitamin D or a salt, ester, or amide suitable for human ingestion thereof; vitamin E or a salt, ester, or amide suitable for human ingestion thereof; vitamin K or a salt, ester, or amide suitable for human ingestion thereof; vitamin O or a salt, ester, or amide suitable for human ingestion thereof; wafer ash; wahoo; wallflower; wasabi; water avens; water dock; water hemlock; watercress; wheat bran; wheatgrass; whey protein; white dead nettle flower; white hellebore; white horehound; white lily; white mulberry; white mustard; white oak; white pepper; white sandalwood; wild carrot; wild cherry; wild daisy; wild indigo; wild lettuce; wild mint; wild thyme; wild yam; willard water; willow bark; wine; winter cherry; winter savory; wintergreen; witch hazel; wood anemone; wood sage; wood sorrel; wormseed; wormwood; xanthan gum; Xanthoparmelia; xylitol; yarrow; yellow dock; yellow loosestrife; yellow toadflax; yerba mansa; yerba mate; yerba santa; yew; yin chen; ylang ylang oil; yogurt; yohimbe; yucca; zeaxanthin; zedoary; zinc; and zizyphus.

Compositions and/or formulations of the present invention may be administered in any form, including one of a capsule, a cachet, a pill, a tablet, a powder, a granule, a pellet, a bead, a particle, a troche, a lozenge, a pastille, a solution, an elixir, a syrup, a tincture, a suspension, an emulsion, a mouthwash, a spray, a drop, an ointment, a cream, a gel, a paste, a transdermal patch, a suppository, a pessary, cream, a gel, a paste, a foam, and combinations thereof for example. Compositions and/or formulations of the present invention may also include an acceptable additive (e.g. one of a solubilizer, an enzyme inhibiting agent, an anticoagulant, an antifoaming agent, an antioxidant, a coloring agent, a coolant, a cryoprotectant, a hydrogen bonding agent, a flavoring agent, a plasticizer, a preservative, a sweetener, a thickener, and combinations thereof) and/or an acceptable carrier (e.g. one of an excipient, a lubricant, a binder, a disintegrator, a diluent, an extender, a solvent, a suspending agent, a dissolution aid, an isotonization agent, a buffering agent, a soothing agent, an amphipathic lipid delivery system, and combinations thereof).

Implementations of the water-soluble form of ginger extract may conveniently be presented in unit dosage form. Unit dosage formulations may be those containing a daily dose or unit, a daily sub-dose, or an appropriate fraction thereof, of the administered components as described herein.

A dosage unit may include the water-soluble form of ginger extract admixed with a second pharmaceutically acceptable additive (in additive to the non-aqueous liquid used in the preparation of the water-soluble form of ginger extract).

The dosage for oral use of the water-soluble form of ginger extract can be from 1 to 30,000 mg per day, split into one or more dosages. For exercise performance enhancing purposes, the formulation is administered (for example, by ingestion) prior to exercise, for example any time between 3 days prior to exercise and right before exercise. This dosing schedule is unexpected, because current literature indicates that for ginger to be effective against DOMS, multiple doses must be taken for days or even weeks before exercise in order to have an effect (Compare Matsumura et al., “The Effects of Pre-Exercise Ginger Supplementation on Muscle Damage and Delayed Onset Muscle Soreness,” Phytother Res. 2015, 29 (6): 887-93; and Contreras, “The Effects of Ginger Supplementation on Delayed Onset Muscle Soreness,” 2018 Kinesiology Student Research from the University of Texas at Arlington accessible https://mavmatrix.uta.edu/kinesiology_studentwork/77/). Contreras found that acute administration of ginger at 2 grams one hour before exercise was ineffective in reduce DOMS. Accordingly, in some aspects, the oral use of the water-soluble form of ginger extract may be administered even a week before exercise. In some aspects, the formulation is administered between 24 hours to immediately before exercise. In certain implementations, the formulation is administered one (1) hour before exercise.

The dosage units may be in a form suitable for administration by standard routes. In general, the dosage units may be administered, by non-limiting example, by topical (including buccal and sublingual), transdermal, oral, rectal, ophthalmic (including intravitreal or intracameral), nasal, vaginal, and/or parenteral (including subcutaneous, intramuscular, intravenous, intradermal, intratracheal, and epidural) routes.

For the exemplary purposes of this disclosure, oral delivery may be a particularly advantageous delivery route for administration to humans and animals of implementations of a pharmaceutical composition, optionally formulated with appropriate pharmaceutically acceptable additives to facilitate administration.

It should be appreciated that any of the components of particular implementations of the water-soluble form of ginger extract may be used as supplied commercially, or may be preprocessed by, by non-limiting example, any of the methods and techniques of agglomeration, air suspension chilling, air suspension drying, balling, coacervation, comminution, compression, pelletization, cryopelletization, extrusion, granulation, homogenization, inclusion compoundation, lyophilization, melting, mixed, molding, pan coating, solvent dehydration, sonication, spheronization, spray chilling, spray congealing, spray drying, or other processes known in the art depending in part on the dosage form desired. The various components may also be pre-coated or encapsulated as known in the art. It will also be clear to one of ordinary skill in the art that appropriate additives may also be introduced to the composition or during the processes to facilitate the preparation of the dosage forms, depending on the need of the individual process.

Those of ordinary skill in the art will be able to readily select manufacturing equipment and pharmaceutically acceptable additives or inert ingredients to manufacture implementations of the water-soluble form of ginger extract. For the exemplary purposes of this disclosure, some examples of pharmaceutically acceptable additives or inert ingredients and manufacturing process are included below, particularly those that relate to manufacture of implementations of the water-soluble form of ginger extract in tablet form. Notwithstanding the specific examples given, it will be understood that those of ordinary skill in the art will readily appreciate how to manufacture implementations of the water-soluble form of ginger extract according to the other methods of administration and delivery disclosed in this document.

A particular implementation of the water-soluble form of ginger extract may include a lubricant. Lubricants are any anti-sticking agents, glidants, flow promoters, and the like materials that perform a number of functions in tablet manufacture, for example, such as improving the rate of flow of the tablet granulation, preventing adhesion of the tablet material to the surface of the dies and punches, reducing interparticle friction, and facilitating the ejection of the tablets from the die cavity. Lubricants may comprise, for example, magnesium stearate, calcium stearate, talc, and colloidal silica.

Particular implementations of the water-soluble form of ginger extract may also include a binder. Binders are any agents used to impart cohesive qualities to powdered material through particle-particle bonding. Binders may include, for example, matrix binders (e.g., dry starch, dry sugars), film binders (e.g., celluloses, bentonite, sucrose), and chemical binders (e.g., polymeric cellulose derivatives, such as methyl cellulose, carboxy methyl cellulose, and hydroxy propyl cellulose); and other sugar, gelatin, non-cellulosic binders and the like.

Disintegrators may be used in particular implementations of the water-soluble form of ginger extract to facilitate the breakup or disintegration of tablets after administration. Disintegrators may include, for example, starch, starch derivatives, clays (e.g., bentonite), algins, gums (e.g., guar gum), cellulose, cellulose derivatives (e.g., methyl cellulose, carboxymethyl cellulose), croscarmellose sodium, croscarmellose cellulose, and other organic and inorganic materials.

Implementations of the water-soluble form of ginger extract may include diluents, or any inert substances added to increase the bulk of the water-soluble form of ginger extract to make a tablet a practical size for compression. Diluents may include, for example, calcium phosphate, calcium sulfate, lactose, mannitol, magnesium stearate, potassium chloride, and citric acid, among other organic and inorganic materials.

Buffering agents may be included in the water-soluble form of ginger extract and may be any one of an acid and a base, where the acid is, for example, propionic acid, p-toluene sulfonic acid, salicylic acid, stearic acid, succinic acid, tannic acid, tartaric acid, thioglycolic acid, or toluene sulfonic acid, and the base is, for example, ammonium hydroxide, potassium hydroxide, sodium hydroxide, sodium hydrogen carbonate, aluminum hydroxide, calcium carbonate, and other organic and inorganic chemicals.

Implementations of the water-soluble form of ginger extract may also be administered through use of amphipathic lipid delivery systems (such as liposomes and unilamellar vesicles), caplet systems, oral liquid systems, parenteral and/or intravenous systems, topical systems (creams, gels, transdermal patches, etc.), intranasal systems, rectal or vaginal systems, and many other delivery methods and/or systems known to those of ordinary skill in the art. Those of ordinary skill in the art will readily be able to select additional pharmaceutically acceptable additives to enable delivery of implementations of a pharmaceutical composition from the disclosure in this document.

With respect to delivery of particular implementations of the water-soluble form of ginger extract, for the exemplary purposes of this disclosure, softgels may be utilized. A softgel is an oral dosage form for medicine in the form of a specialized capsule. They consist of a shell surrounding a liquid fill. Softgels can allow for near instant release of the solution in the G.I. tract, ensuring rapid onset of action as well as enhanced bioaccesibility and bioavailability.

Tablets and other orally discrete dosage forms, such as capsules, cachets, pills, granules, pellets, beads, and particles, for example, may optionally be coated with one or more enteric coatings, seal coatings, film coatings, barrier coatings, compress coatings, fast disintegrating coatings, or enzyme degradable coatings for example. Multiple coatings may be applied for desired performance. Further, dosage forms may be designed for, by non-limiting example, immediate release, pulsatile release, controlled release, extended release, delayed release, targeted release, synchronized release, or targeted delayed release. For release/absorption control, carriers may be made of various component types and levels or thicknesses of coats. Such diverse carriers may be blended in a dosage form to achieve a desired performance. In addition, the dosage form release profile may be affected by a polymeric matrix composition, a coated matrix composition, a multi-particulate composition, a coated multi-particulate composition, an ion-exchange resin-based composition, an osmosis-based composition, or a biodegradable polymeric composition.

While manufacture of implementations of the water-soluble form of ginger extract have been described in particular sequences of steps and/or in particular forms in the examples, it will be understood that such manufacture is not limited to the specific order of steps or forms as disclosed. For example, a different solvent than water in which the extract of ginger and the source of lithium is readily soluble and which can be evaporated can be used. Any steps or sequences of steps of manufacture of implementations of the water-soluble form of ginger extract in any form are given as examples of possible steps or sequences of steps or potential forms and not as limitations, since many possible manufacturing processes and sequences of steps may be used to manufacture water-soluble form of ginger extract implementations in a wide variety of forms.

In other aspects, the disclosure relates to methods of treating or alleviating a ginger-treatable ailment in a subject. For simplicity purposes, the conditions and diseases that can be treated by administration of ginger extract or its isolated compounds will be referred to as “ginger-treatable ailments”. The method of treating a ginger-treatable ailment comprises administering the water-soluble form of ginger extract described herein to a subject.

As used herein, the term “disease” refers to a state of health of an animal (including humans) wherein the animal cannot maintain homeostasis, and wherein if the disease is not ameliorated then the animal's health continues to deteriorate. In contrast, as used herein, the term “disorder” in an animal refers to a state of health in which the animal (including humans) is able to maintain homeostasis, but in which the animal's state of health is less favorable than it would be in the absence of the disorder. Left untreated, a disorder does not necessarily cause a further decrease in the animal's state of health.

The “ginger-treatable ailments” include, but are not limited to, exercise-induced muscle soreness, motion sickness, acute respiratory distress syndrome (ARDS), allergic rhinitis (hay fever), antiretroviral-induced nausea and vomiting, asthma, back pain, burns, cancer induced anorexia, chemotherapy induced vomiting and nausea, chronic obstructive pulmonary disorder, colic, constipation, Coronavirus disease 2019 (COVID-19), diabetes (both type I and II), diarrhea, dry mouth, dyspepsia, erectile dysfunction, gastroenteritis-related nausea and vomiting, hangover, hyperlipidemia, hypertension, hypothyroidism, insect bites, intraoperative nausea and vomiting, irritable bowel syndrome, joint pain, menopausal symptoms, migraine, headaches, multiple sclerosis, non-alcoholic fatty liver disease, obesity, parturition, polycystic ovary syndrome, postoperative nausea and vomiting, postoperative recovery, postpartum complications, rheumatoid arthritis, toxin induced liver damage, traumatic brain injury, ulcerative colitis, respiratory tract infections, vertigo, and herpes simplex infection.

A disease or disorder is “alleviated” if the severity of a symptom of the disease or disorder, the frequency with which such a symptom is experienced by a patient, or both, is reduced.

A disease is “cured” if the disease no longer shows any symptoms, and appropriate diagnostic tests show absence of it on a patient.

As used herein, “treat,” “treatment,” and “treating,” a disease or disorder means reducing the severity and/or frequency, with which a sign and/or symptom of the disease or disorder is experienced by a subject. The subject may be symptomatic or asymptomatic at the time of treatment. In other words, “treat,” “treatment,” and “treating,” can be to reduce, ameliorate or eliminate signs or symptoms associated with a condition present in a subject, or can be metaphylactic or prophylactic, (i.e. to prevent or reduce the occurrence of the symptoms in a subject, or to delay onset of possible or expected signs or symptoms in a subject). Such prophylactic or metaphylactic treatment can also be referred to as prevention of the condition.

In other aspects, this disclosure discusses formulations and methods to treat the “ginger-treatable conditions” mentioned above. The symptoms and etiology of the above conditions that can be ameliorated by administration of the formulations described herein can be found in the book “Handbook of Diseases 3rd Edition” by Springhouse Corporation, fully incorporated here by reference.

Implementations of the water-soluble form of ginger extract are particularly useful in increasing athletic performance in a subject. In some aspects, the water-soluble form of ginger extract decreases muscle soreness during or after the exercise (the latter being commonly as DOMS). This result is highly surprising, as multiple studies have shown ginger to have no effect on preventing or decreasing muscle soreness during exercise.

### EXAMPLES

The present disclosure is further illustrated by the following examples that should not be construed as limiting. The contents of all references, patents, and published patent applications cited throughout this application, as well as the Figures, are incorporated herein by reference in their entirety for all purposes.

### Example 1

In 300 ml of water placed in an ice bath, gradually dissolve the source of lithium (metallic lithium at 15 g or lithium hydroxide at 49 g)) while making sure the temperature of the solution does not to rise near the boiling point (100° C.). This is crucial when the source of lithium is metallic lithium, as the reaction of the uncharged form of lithium metal with water is extremely exothermic. If any water is lost due to heat during this step, more water should be added so as to bring the total liquid volume back to ˜300 ml. The time required to dissolve about 50 g of lithium hydroxide will be slow even with stirring. If small pieces of the lithium hydroxide remain undissolved in the solution, it would be fine to proceed to the next step, as those small pieces of lithium will generally dissolve once the water solution is mixed with ginger extract and propylene glycol.

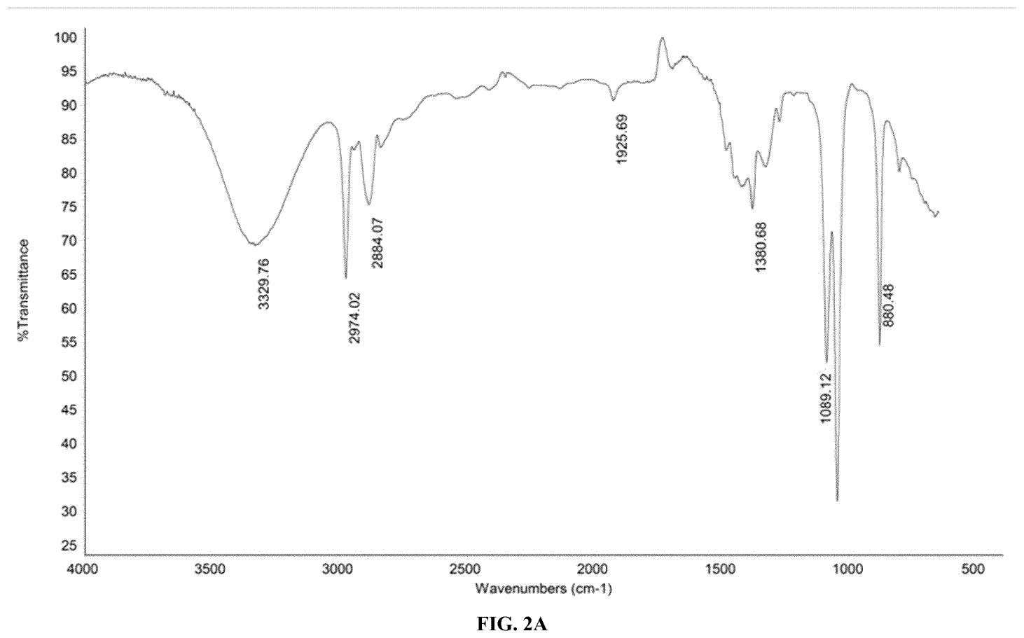

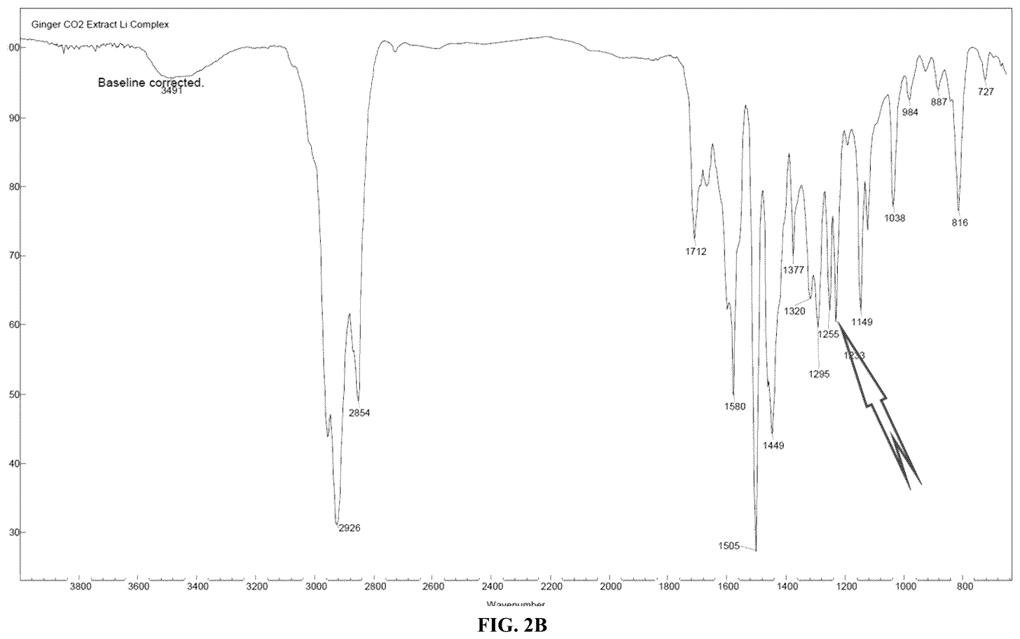

A volume of 1000 ml ginger extract (total CO2 liquid ginger extract) is then added to the water solution. The resulting mixture already exhibits excellent water solubility. Afterwards, a volume of 1000 ml propylene glycol is then added to the water solution to produce a water-soluble form of ginger extract (FIG. 2A) that is also stable in capsules. The resulting solution displays excellent solubility in water (>100 mg/ml, see FIG. 2B) as well as in acidic simulated gastric fluid and slightly alkaline solutions (pH 7-9).

The resulting solution exhibited excellent stability characteristics, remaining stable for over 6 months at room temperature.

On the contrary, none of the methods used to commonly separate an emulsion such as salting out, sonification, and centrifugation led to any appreciable separation of the total CO2 liquid ginger extract solution.

### Example 2

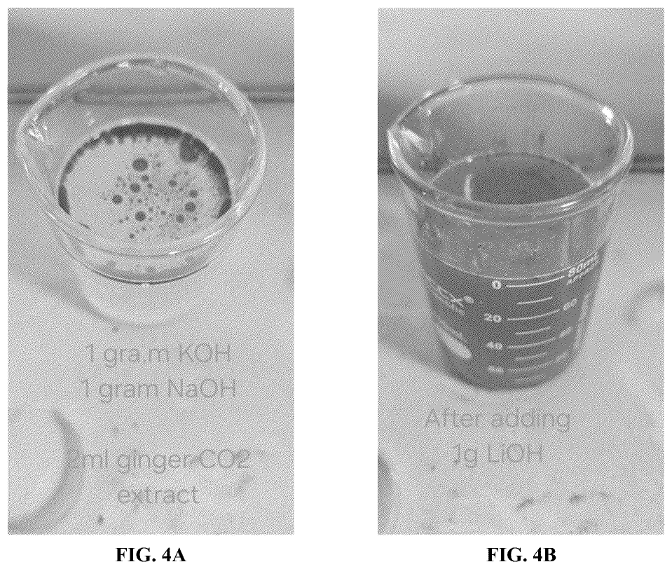

To further distinguish that the water solubility of the ginger extract gained through the disclosed method is due to unique unexpected properties of lithium and not merely due to exposing the ginger extract to alkaline pH, reactions with sodium hydroxide and potassium hydroxide were tested. In 100 ml distilled water, 1 gram NaOH and 1 gram KOH were dissolved. Afterwards, 2 ml of total CO2 liquid ginger extract were added to the mixture. The aqueous solution exhibited a light yellowish tint, but the ginger extract noticeably remained undissolved floating on the top even after 1 hour (FIG. 4A). After this period of observation, 1 gram LiOH was then added to the solution. After 2 minutes and without any stirring, all the ginger extract had dissolved giving the aqueous solution a bright orange color (FIG. 4B).

### Example 3

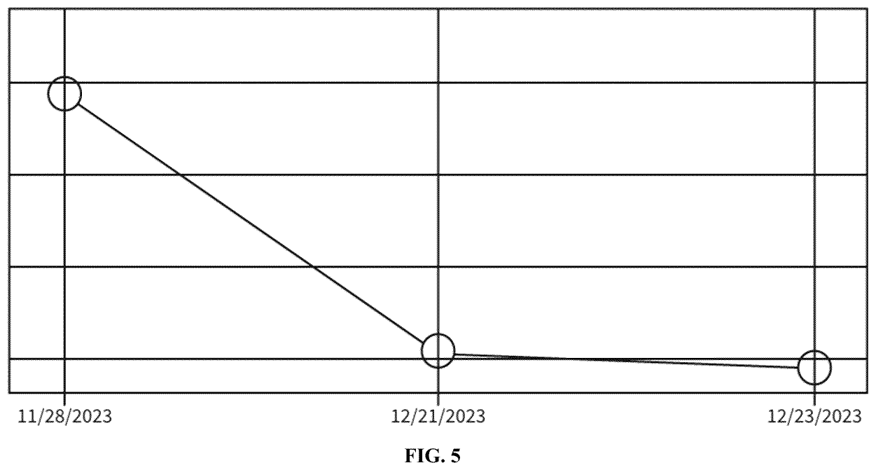

A 42-year-old man suffering from joint pain started to ingest the preparation produced in Example 1 at a dose of 1 ml twice per day between meals. His initial CRP (C-reactive protein, a protein that increases with systemic inflammation) was abnormally high prior to ingesting the water-soluble form of ginger extract. Following 3 weeks of ingesting the preparation produced in Example 1 (one 000 capsule twice per day), his CRP levels decreased dramatically (FIG. 5). His CRP levels continued to reduce even 1 week later. Interestingly, lithium itself has no appreciable effects on CRP levels. One of the most recent meta-analyses on the long term effects of ginger supplementation on CRP levels showed a moderate decrease by ˜2 mg/L from ginger supplementation (duration 10 weeks-3 months and various ginger doses from 1-3 grams/day, results in FIG. 2 of Mazidi et al., “The effect of ginger supplementation on serum C-reactive protein, lipid profile and glycaemia: a systematic review and meta-analysis,” Food & Nutrition Research, 2016, 60:1).

The subject's pain dramatically decreased from the first dose in mere minutes after ingestion. His pain level remained significantly lower through the therapy with the water-soluble form ginger extract.

### Example 4

A 40-year-old female subject, who is an avid runner and swimmer, suffered from aches and pains in her back, neck, and shoulders from a slip and fall accident. The pain did not subside after several chiropractor, acupuncture, and cupping treatments. However, within 10 min of drinking 2 ml of the preparation produced in Example 1 with 8 oz of water, she reported all aches and pains were instantly gone. The subject also reported a feeling of enhanced energy lasting at least 2.5 hours after ingestion of the preparation.

### Example 5

A 59-year-old male subject with severe knee and back pain had no pain relief with conventional pain management therapeutics. When he ingested capsules contain a total of 2 ml of the preparation produced in Example 1, the subjected reported cessation of pain for several hours.

### Example 6

Two females (34 years old and 41 years old) with a chronic herpes simplex virus infection with frequent outbreaks of blisters on their lips ingested 1000 ml of the water-soluble ginger extract produced according to Example 1 while acutely suffering from large painful blisters. Within minutes of taking the water-soluble ginger extract, they felt immediate relief of the symptoms. After a few hours, the blisters had been greatly reduced in size with a notable reduction in pain and discomfort. By the next day, the blisters had practically disappeared.

For past outbreaks, both subjects tried multiple remedies, both pharmaceutical and natural, to treat the blisters. They include Abriva®, tea tree oil, toothpaste, lysine, acyclovir, topical hydrogen peroxide, Vaseline® and Aquafor®. None of those remedies provided any noticeable immediate relief or greater healing of the symptoms.

### Example 7

A 43-year-old male performed repeated knee extension exercise at 70% maximum weight until he had to stop due to muscle soreness (“burn”). His quadriceps exhibited pain for the next 3 days. After one week, he repeated the exercise one hour after ingesting 1000 ml of the water-soluble ginger extract produced according to Example 1 and managed repetitions until he had to stop due to muscle soreness. His quadriceps exhibited no pain the next day.

### Example 8

A 43-year-old male ingested 2.5 grams of conventional total CO2 liquid ginger extract in the form of capsules during the morning with empty stomach. During the rest of the day he experienced no notable burning during urination or any smell of ginger in his urine. After a 5-day washout period, he ingested 5 grams of the water-soluble ginger extract (which was prepared to according to the process of Example 1 but using 2.5 grams total CO2 liquid ginger extract). Upon subsequent urinations during the day, he felt a burning sensation while his urine had a distinct smell of ginger. This shows a much greater amount of ginger was absorbed from ingestion of the water-soluble ginger extract, which had to be subsequently excreted in urine.

### Example 9

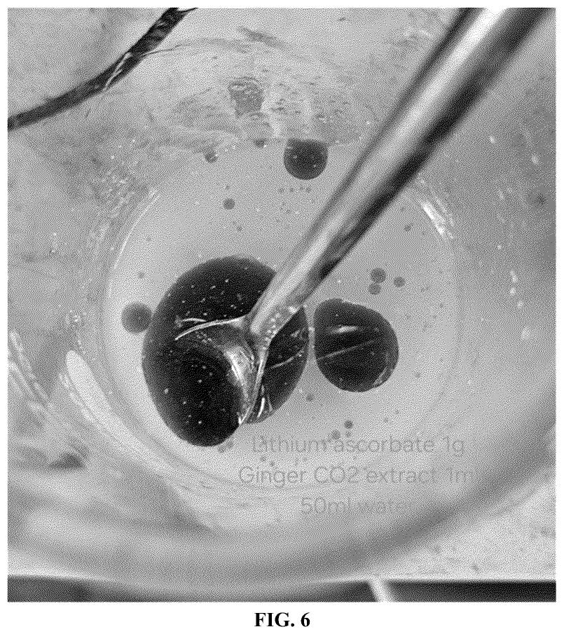

In 3 different beakers 50 ml of distilled water and 1 ml of total CO2 liquid ginger extract were added. To each beaker were added 1 gram of either lithium citrate, lithium ascorbate or lithium lactate. The ginger extract, even after stirring, exhibited no notable dissolution in the water (FIG. 6).

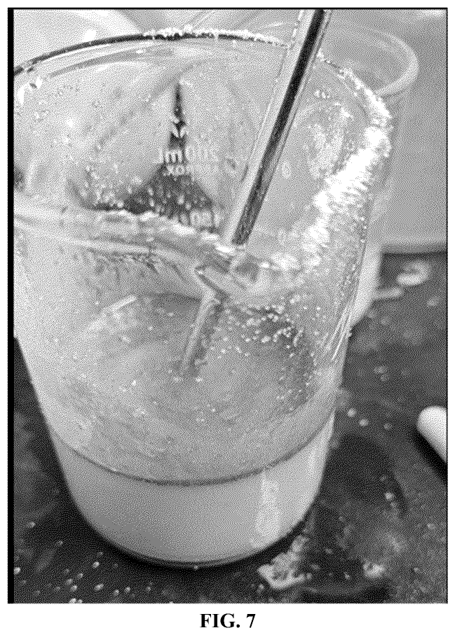

When 1 gram of LiOH was added to the solution, the ginger CO2 extract dissolved totally in the water. To further elucidate whether the lithium forms complexes with actives of the ginger CO2 extract is pH-independent, 2 ml of the extract prepared as per Example 1 were dissolved in 100 ml of water. The solution was acidified by addition of 5 grams of citric acid. Although a small amount of the extract floated out of the solution, the vast majority of it remained dissolved (FIG. 7).

1. A composition for human ingestion comprising:
an extract of ginger; and
a source of lithium.

2. The composition of claim 1, wherein the source of lithium is metallic lithium.

3. The composition of claim 1, wherein the source of lithium is charged lithium.

4. The composition of claim 3, where the source of charged lithium is lithium hydroxide, lithium hydride, or a water-soluble salt of lithium with a weak acid.

5. The composition of claim 1, further comprising water.

6. The composition of claim 1, wherein the composition is a solution, and the pH of the solution is 8-14.

7. The composition of claim 1, wherein the weight ratio of the source of lithium and the extract of ginger is between about 1:100 and about 100:1.

8. The composition of claim 1, wherein the composition comprises an amount of lithium as either uncharged or ionic form in the composition that is at least 0.1% by weight.

9. The composition of claim 1, wherein the concentration of active compounds in the composition is at least about 1000 micromole/L.

10. The composition of claim 1, additionally comprising a dietary ingredient, a food additive, or a pharmaceutically acceptable additive.

11. The composition of claim 10, wherein the pharmaceutically acceptable additive is polyethylene glycol.

12. The composition of claim 11, wherein pharmaceutically acceptable additive is PEG 400.

13. The composition of claim 1, wherein the composition's solubility in water is at least about 10 mg/L.

14. The composition of claim 1, wherein the composition is in the form of a tablet, capsule, a solution, an elixir, a syrup, a tincture, a suspension, an emulsion, a mouthwash, a spray, a drop, an ointment, a cream, a gel, a paste, a suppository, a pessary, a cream, a gel, or a foam.

15. The composition of claim 1, wherein the composition is in the form of a softgel.

16. A method of treating a ginger treatable ailment in a subject comprising administering to the subject the composition of claim 6, wherein the subject is administered about 0.01-30,000 ml of the composition per day.

17. The method of claim 16, wherein the subject is administered the composition in a softgel formulation.

18. The method of claim 16, wherein the ginger treatable ailment is herpes simplex virus.

19. The method of claim 16, wherein the subject is exhibiting an outbreak in the form of mouth sores, and the administration of the composition per day results in reduced size of the sores and the pain and swelling associated with them.

20. The method of claim 16, where the subjects experiences alleviation of the symptoms in 2 hours or less after ingesting the water-soluble ginger extract.

21. A method of improving athletic performance in a subject, the method comprising administering to the subject the composition of claim 6, wherein the subject is administered 0.01-30,000 ml of the composition prior to the subject exercising.

22. The method of claim 21, wherein improvement in athletic performance manifests as reduced muscle soreness during and/or after exercise.

23. A method of improving bioaccesibility, solubility, and/or bioavailability of an extract of ginger, said method comprising:
providing the extract of ginger; and
mixing a source of lithium with the extract of ginger, thereby producing a water-soluble form of ginger extract.

24. The method of claim 23, wherein the source of lithium is selected from the group consisting of: metallic lithium, lithium hydroxide, lithium hydride, and a water-soluble salt of lithium with a weak acid.

25. The method of claim 24, further comprising dissolving the source of lithium in water to produce a lithium solution, wherein the lithium solution is mixed with the ginger extract.

26. The method of claim 23, further comprising mixing at least one non-aqueous liquid with the water-soluble form of ginger extract.

27. The method of claim 26, wherein the at least one non-aqueous liquid is selected from the group consisting of: a polyol, an alcohol, or an oil.

28. The method of claim 27, wherein the at least one non-aqueous liquid is selected the group consisting of polyethylene glycol (PEG), glycerol, and propylene glycol.

29. The method of claim 28, wherein the at least one non-aqueous liquid is PEG 400.

30. The method of claim 25, wherein the weight ratio of the source of lithium and the extract of ginger mixed together is between about 1:100 and about 100:1.
